(19) United States
(12) Patent Application Publication
(10) Pub. No.: US 2021/0096138 A1
(43) Pub. Date: Apr. 1, 2021

(54) HISTOLOGY GUIDED MASS SPECTROMETRY

(71) Applicants: Katy Ryan Smoot, Morgantown, WV (US); Matthew Jacob Powell, Westover, WV (US); Joerg Manfred Steiner, College Station, TX (US); Sina Marsilio, Davis, CA (US)

(72) Inventors: Katy Ryan Smoot, Morgantown, WV (US); Matthew Jacob Powell, Westover, WV (US); Joerg Manfred Steiner, College Station, TX (US); Sina Marsilio, Davis, CA (US)

(21) Appl. No.: 16/588,047

(22) Filed: Sep. 30, 2019

(51) Int. Cl. G01N 33/68; H01J 49/16; H01J 49/00

(52) U.S. Cl. CPC G01N 33/6851; G01N 2800/065; H01J 49/0036; H01J 49/164

(57) ABSTRACT

The use of matrix-assisted laser desorption ionization (MALDI) mass spectrometry imaging (MSI) to assay feline tissue samples is described. Methods of analyzing a tissue sample may generally comprise generating sample ions directly from the tissue sample using a MALDI ionization source, receiving the ions into a mass spectrometer, identifying at least one inflammatory bowel disease and/or lymphoma related compound in the sample from results from the mass spectrometer, comparing the at least one identified related compound in the sample to one or more known inflammatory bowel disease and/or lymphoma profiles, and identifying at least one condition related to the sample from the comparison of the at least one identified related compound to the one or more known inflammatory bowel disease and/or lymphoma profiles. The mass spectrometer may be a quadrupole mass spectrometer, a time of flight mass spectrometer, an Orbitrap mass spectrometer, or an ion trap mass spectrometer.

### TECHNICAL FIELD

The present invention relates to histology guided mass spectrometry, and in particular, systems and methods of using a matrix-assisted laser desorption/ionization time-of-flight mass spectrometer (MALDI-TOF MS) to analyze histologic tissue samples for feline inflammatory bowel disease and feline lymphoma.

### BACKGROUND

Inflammatory bowel disease (IBD) and lymphoma are common diseases of the feline gastrointestinal system. IBD includes a group of immunologically mediated disorders of the digestive tract, commonly presenting as a chronic, lymphoplasmacytic enteritis (LPE) affecting the small intestine. Gastrointestinal (alimentary) lymphoma is the most common clinical presentation of lymphoma in cats. The vast majority of feline alimentary lymphomas are histologically classified as intestinal small cell lymphoma (ISCL). Other less frequent types of feline alimentary lymphomas include large B-cell lymphoma and large granular lymphocyte lymphoma.

Differentiating IBD from ISCL in cats using conventional physical, ultrasonographic, endoscopic, and/or microscopic examinations may be extremely difficult. Both conditions are commonly diagnosed in middle aged to older cats of any breed and sex, and IBD and ISCL may coexist in the same cat. The most common clinical signs with both diseases include vomiting, diarrhea, weight loss and changes in appetite. In addition, systemic diseases, such as endocrine, renal, or hepatic disease, dietary sensitivity, and infectious diseases may cause similar symptoms. Even differentiating feline IBD and ISCL on the basis of endoscopically obtained biopsy samples may be difficult.

Matrix-assisted laser desorption ionization (MALDI) mass spectrometry imaging (MSI) may be used to analyze metabolites, peptides and proteins, DNA segments, and lipids directly from tissue sections with spatial fidelity. MALDI MSI may be performed on either fresh frozen or formalin-fixed, paraffin-embedded (FFPE) tissue specimens. MSI may be used to elucidate molecular profiles of different diseases. Conventional MSI may suffer from one or more of the following limitations: spatial resolution, mass accuracy, spectral resolution, sensitivity, robustness, reproducibility, requirements for sample preparation, and degree of technical difficulty.

Accordingly, more efficient mass spectrometry imaging systems and methods of making and using the same are desirable.

### SUMMARY

This disclosure generally relates to the use of matrix-assisted laser desorption ionization (MALDI) mass spectrometry imaging (MSI) to assay feline tissue samples. Methods of analyzing a feline tissue sample may generally comprise generating sample ions directly from the feline tissue sample using a MALDI ionization source, receiving the ions into a mass spectrometer, identifying at least one feline inflammatory bowel disease (IBD) related compounds and feline lymphoma related compounds in the feline tissue sample from results from the mass spectrometer, comparing the at least one identified related compound in the feline tissue sample to one or more known feline IBD profiles and/or feline lymphoma profiles, and identifying at least one condition related to the feline tissues sample from the comparison of the at least one identified related compound to the one or more known profiles.

### BRIEF DESCRIPTION OF THE DRAWINGS

The present invention described herein may be better understood by reference to accompanying figures, in which:

FIG. 1 shows a histology guided mass spectrometry profiling workflow according to the present invention;

FIG. 2 shows a mass spectrum taken from feline tissue samples according to the present invention;

FIG. 3 shows the probability that subjects having a positive screening test for IBD or ISCL have the disease according to the present invention;

FIG. 4 shows the probability that subjects having a negative screening test for IBD or ISCL lack the disease according to the present invention;

FIG. 5 shows the data distribution for test profiles and reference profiles according to the present invention; and

FIG. 6 shows the data of a validation study profile according to the present invention.

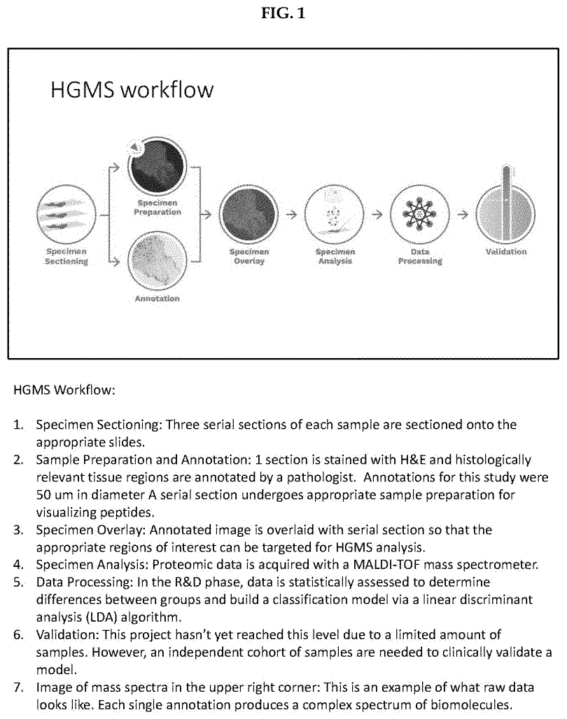

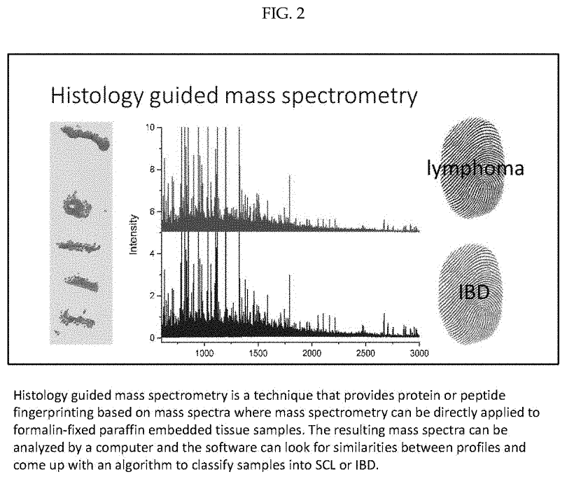

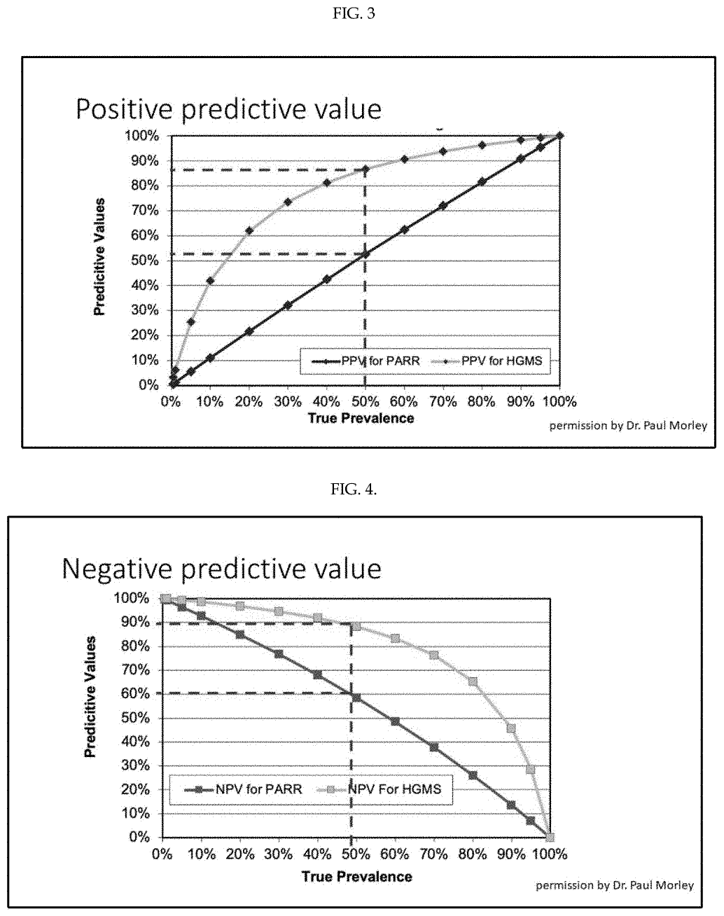

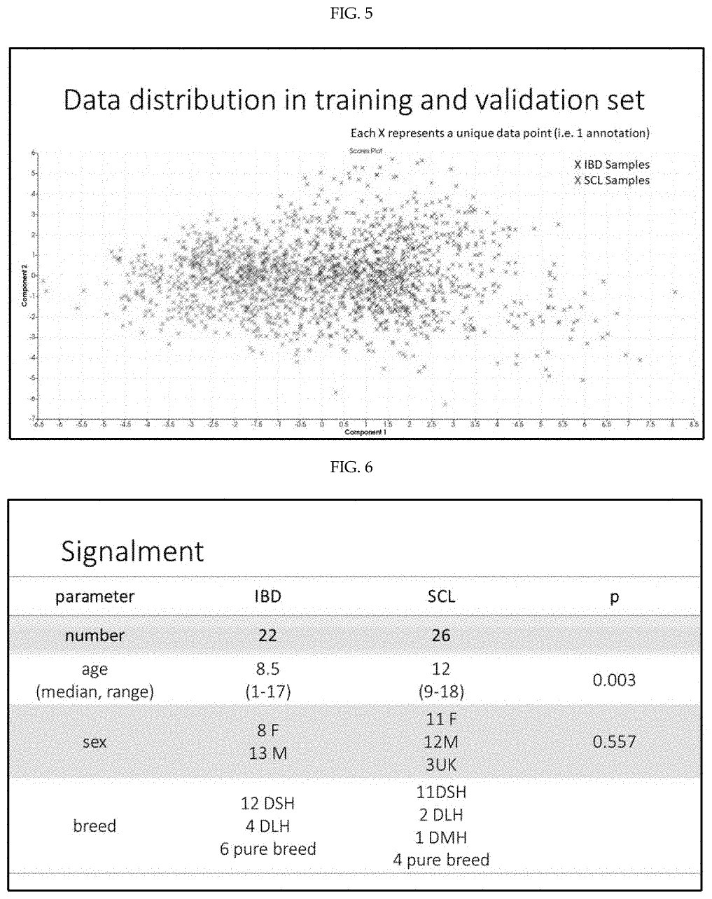

### DETAILED DESCRIPTION

As generally used herein, the articles “the”, “a”, and “an” refer to one or more of what is claimed or described.

As generally used herein, the terms “include”, “includes”, and “including”, “have”, “has”, and “having”, and “characterized by” are meant to be non-limiting.

As generally used herein, the term “about” refers to an acceptable degree of error for the quantity measured, given the nature or precision of the measurements. Typical exemplary degrees of error may be within 20%, 10%, or 5% of a given value or range of values. Alternatively, and particularly in biological systems, the terms “about” refers to values within an order of magnitude, potentially within 5-fold or 2-fold of a given value.

All numerical quantities stated herein are approximate unless stated otherwise. Accordingly, the term “about” may be inferred when not expressly stated. The numerical quantities disclosed herein are to be understood as not being strictly limited to the exact numerical values recited. Instead, unless stated otherwise, each numerical value is intended to mean both the recited value and a functionally equivalent range surrounding that value. At the very least, and not as an attempt to limit the application of the doctrine of equivalents to the scope of the claims, each numerical parameter should at least be construed in light of the number of reported significant digits and by applying ordinary rounding techniques. Notwithstanding the approximations of numerical quantities stated herein, the numerical quantities described in specific examples of actual measured values are reported as measured.

Any numerical range recited in this specification is intended to include all sub-ranges of the same numerical precision subsumed within the recited range. For example, a range of “1.0 to 10.0” or “1.0-10.0” is intended to include all sub-ranges between (and including) the recited minimum value of 1.0 and the recited maximum value of 10.0, that is, having a minimum value equal to or greater than 1.0 and a maximum value equal to or less than 10.0. Any maximum numerical limitation recited in this disclosure is intended to include all lower numerical limitations subsumed therein and any minimum numerical limitation recited in this disclosure is intended to include all higher numerical limitations subsumed therein. Accordingly, Applicants reserve the right to amend this specification, including the claims, to expressly recite any sub-range subsumed within the ranges expressly recited herein.

This disclosure describes various features, aspects, and advantages of various aspects of the invention. It is understood, however, that this disclosure embraces numerous alternative features, aspects, and/or advantages that may be accomplished by combining any of the various features, aspects, and advantages of the various embodiments described herein in any combination or sub-combination that one of ordinary skill in the art may find useful. Such combinations or sub-combinations are intended to be included within the scope of this disclosure. As such, the claims may be amended to recite any features, aspects, and advantages expressly or inherently described in, or otherwise expressly or inherently supported by, this disclosure. Further, any features, aspects, and advantages that may be present in the prior art may be affirmatively disclaimed. Accordingly, this disclosure may comprise, consist of, or consist essentially of or be characterized by one or more of the features, aspects, and advantages described herein.

In the following description, certain details are set forth in order to provide a better understanding of various features, aspects, and advantages the invention. However, one skilled in the art will understand that these features, aspects, and advantages may be practiced without these details and/or in the absence of any details not described herein. In other instances, well-known structures, methods, and/or techniques associated with methods of practicing the various features, aspects, and advantages may not be shown or described in detail to avoid unnecessarily obscuring descriptions of other details of the various embodiments.

As generally used herein, the term “proteome” refers to the entire complement of proteins produced by an organism or biological system, including modifications made to particular proteins. The proteome of an organism may vary with time, and may also depend on the various stresses that the organism or biological system undergoes. As generally used herein, the term “proteomic profile” refers to information about the protein content of a sample, as characterized by peaks in its mass spectrum corresponding to biomarkers such as proteins, glycoproteins, glycopeptides, peptidoglycans, and other biological substances making up the proteome. The proteomic profile may include all or part of the information, for example, mass spectrometric data, such as m/z values of peaks in the mass spectrum. The proteomic profile may comprise peptide peaks from enzymatic digestion. The proteomic profile may comprise peptidoglycans and/or glycans from enzymatic digestion. For example, the proteomic profile may comprise all or substantially all digested proteins and/or lack the entire complement of proteins. It is also possible that substances that are not proteins may be represented in the mass spectrum, for example carbohydrates or lipo-polysaccharides, as well as endogenous peptides, glycoproteins, glycopeptides, peptidoglycans, and other substances as mentioned above. For the sake of simplicity, however, the term “proteomic profile” and “molecular profile” may be used interchangeably herein and each being represented by the peaks in the mass spectra.

As generally used herein, the terms “subject”, “individual”, and “patient” are used interchangeably herein and refer to any mammalian subject, particularly cats. Other subjects may include humans, cattle, dogs, guinea pigs, rabbits, rats, mice, horses, and so on. In some cases, the methods of the invention may find use in experimental animals, in veterinary application, and in the development of animal models, including, but not limited to, rodents including mice, rats, hamsters, cats, dogs, and primates.

As generally used herein, the term “diagnosis” refers to the determination as to whether a subject is likely affected by a given disease, disorder or dysfunction. The skilled artisan often makes a diagnosis on the basis of one or more diagnostic indicators, i.e., a biomarker, the presence, absence, or amount of which may be indicative of the presence or absence of the disease, disorder or dysfunction. A diagnosis may also include considerations of the grade or stage of a disease, disorder, or dysfunction.

As generally used herein, the term “prognosis” refers to a prediction of the probable course and outcome of a clinical condition or disease, disorder or dysfunction or treatment thereof. A prognosis of a patient is usually made by evaluating factors or symptoms of a disease that are indicative of a favorable or unfavorable course or outcome. For example, a prognosis may be based on one or more diagnostic indicators, i.e., a biomarker, the presence, absence, or amount of which may be indicative of the likely course or outcome of a treatment of a clinical condition or disease, disorder or dysfunction. The prognosis may also be based on the grade or stage of a disease, disorder, or dysfunction. It is understood that the term “prognosis” does not necessarily refer to the ability to predict the course or outcome with 100% accuracy. Instead, the skilled artisan will understand that the term “prognosis” refers to an increased probability that a certain course or outcome will occur; that is, that a course or outcome is more likely to occur in a patient exhibiting a given condition when compared to patients not exhibiting the condition.

As generally used herein, the term “sample” refers to cells and/or tissue that may comprise normal, diseased, or tumor material obtained from a patient. The term encompasses clinical samples, for example, samples obtained by surgical resection and tissue obtained by biopsy, such as for example a core biopsy or a fine needle aspirate biopsy. The term also encompasses samples that may be obtained by techniques such as brush cytology and tissue scrapes. The term also encompasses samples that may be generated by performing flow cytometry on biofluids or cytology samples to concentrate and/or select cells of interest. The term encompasses samples comprising tumor samples obtained fr1111om sites other than the primary tumor, e.g., metastases and circulating tumor cells, as well as well as preserved samples, such as formalin-fixed, paraffin-embedded samples or frozen samples. The term encompasses cells that are the progeny of the patient's tumor cells, e.g., cell culture samples or cell lines derived from primary tumor cells or circulating tumor cells. The term encompasses samples that may comprise protein or nucleic acid material or other biomarkers shed from tumor cells in vivo, e.g., bone marrow, blood, lymph, plasma, serum, and the like. The term also encompasses samples that have been enriched for tumor cells or otherwise manipulated after their procurement and samples comprising polynucleotides and/or polypeptides that are obtained from a patient's tumor material. In the present disclosure, samples may be obtained from esophageal tissue, gastric tissue, the small intestine (duodenum, ileum, and jejunum), the large intestine, junctions between the gastrointestinal organs, other gastrointestinal tissues, lymph nodes, metastatic sites, and biofluids (e.g., blood, urine, lymphatic fluid).

As generally used herein, the term “treatment” refers to administering an agent, or carrying out a procedure (e.g., radiation, a surgical procedure, etc.) to obtain a desired pharmacologic and/or physiologic effect. The effect may be prophylactic in terms of completely or partially preventing a disease or symptom thereof and/or may be therapeutic in terms of effecting a partial or complete cure for a disease and/or symptoms of the disease. The effect may be therapeutic in terms of a partial or complete cure for a disease or condition (e.g., a cancer) and/or adverse effect attributable to the disease or condition. These terms also include any treatment of a condition or disease in a mammal, particularly in a feline, and include: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; (c) relieving the disease, i.e., causing regression of the disease; (d) reducing the severity of a symptom of the disease and/or (e) reducing the frequency of a symptom of the disease or condition.

Without wishing to be bound to any particular theory, there may be a high degree of discordance among experienced veterinarians in a differential diagnosis of feline inflammatory bowel disease and feline lymphoma. Feline inflammatory bowel disease and feline small cell lymphoma may be characterized by having different proteins, and different protein expression levels, and/or different forms of proteins. Frequently, proteins exist in a sample in a plurality of different forms characterized by detectably different masses. These forms may result from either or both of pre- and post-translational modification. Pre-translational modified forms may include allelic variants, splice variants and RNA editing forms. Post-translationally modified forms may include forms resulting from proteolytic cleavage (e.g., fragments of a parent protein), glycosylation, phosphorylation, lipidation, oxidation, methylation, cystinylation, sulphonation and acetylation.

When detecting or measuring a protein in a sample, the ability to differentiate between these different forms may depend upon the nature of the difference and the method used to detect or measure the difference. For example, an immunoassay using a monoclonal antibody may detect all forms of a protein containing the epitope and may not distinguish between them. In diagnostic assays, this inability to distinguish different forms of a protein diminishes the power of the assay. Further, only known biomarkers may be targeted. Thus, it is useful to employ an assay method that distinguishes between forms of a protein and that specifically detects and measures a desired form or forms of the protein. Distinguishing different forms of an analyte or specifically detecting a particular form of an analyte may be referred to as “resolving” the analyte.

The present disclosure relates to more efficient and/or cost-effective systems and methods of using a matrix-assisted laser desorption/ionization time-of-flight mass spectrometer (MALDI-TOF MS) to analyze histologic tissue samples for feline inflammatory bowel disease and feline lymphoma.

The present disclosure describes minimally invasive methods that may be used for differentiating between feline inflammatory bowel disease and feline small cell lymphoma. The methods may include determining whether a proteomic profile of a feline tissue sample from a subject includes at least one characteristic predictive of one of feline inflammatory bowel disease and feline small cell lymphoma.

The present invention is generally directed to mass spectrometry imaging and analysis that may be useful in cases that are histologically equivocal or ambiguous, and a firm prediction of feline inflammatory bowel disease and feline small cell lymphoma cannot be made with absolute certainty. Histology guided mass spectrometry (HGMS) profiling, which is a subset of mass spectrometry imaging, allows for targeted analysis of biomolecules in thin tissue sections from specific cells of interest. HGMS may be used to identify molecular profiles predictive of feline inflammatory bowel disease and feline small cell lymphoma.

Mass spectrometry may be a powerful methodology to detect different forms of a protein because the different forms typically have different masses that may be resolved by the technique (mass spectrometry exploits the intrinsic properties of mass and charge). For example, if one form of a protein is a superior biomarker for a disease over another form of the protein, mass spectrometry may be able to specifically detect and measure the useful form where traditional immunoassay fails to distinguish the forms and fails to specifically detect the useful biomarker.

Mass spectrometry imaging may provide one or more of the following advantages: mass spectrometry works in formalin-fixed paraffin embedded tissue sections; mass spectrometry may provide information to clarify the diagnosis of ambiguous cases by histology; and mass spectrometry is relatively objective, fast, and inexpensive.

The present invention includes a method of differentiating cells or tissues from feline inflammatory bowel disease and feline small cell lymphoma using mass spectrometry analysis. Traditional quantitative mass spectrometry has used electrospray ionization (ESI) followed by tandem mass spectrometry (MS/MS), while newer quantitative and qualitative methods are being developed using matrix assisted laser desorption/ionization (MALDI) followed by time of flight (TOF) mass spectrometry (MS).

In ESI tandem mass spectrometry (ESI/MS/MS), analysis of both precursor ions and product ions is possible, thereby monitoring a single precursor product reaction and producing a signal only when the desired precursor ion is present. Protein quantification has been achieved by quantifying tryptic peptides with the use of isotopically labelled peptide standards. Secondary ion mass spectrometry (SIMS) uses ionized particles emitted from a surface for mass spectrometry at a sensitivity of detection of a few parts per billion. The sample surface is bombarded by primary energetic particles, such as electrons, ions (e.g., O, Cs), neutrals or even photons, forcing atomic and molecular particles to be ejected from the surface, a process called sputtering. Since some of these sputtered particles carry a charge, a mass spectrometer can be used to measure their mass and charge.

Laser desorption mass spectrometry (LD-MS) involves the use of a pulsed laser, which induces desorption of sample material from a sample site effectively. This method may be used in conjunction with a mass spectrometer, and can be performed simultaneously with ionization if one uses the right laser radiation wavelength and/or matrix. When coupled with Time-of-Flight (TOF) measurement, LD-MS is referred to as LDLPMS (Laser Desorption Laser Photoionization Mass Spectrometry). The LDLPMS method of analysis gives instantaneous volatilization (fragmentation) of the sample which permits rapid analysis without any wet extraction chemistry. Signal intensity, or peak height, is measured as a function of travel time. The applied voltage and charge of the particular ion determines the kinetic energy, and separation of fragments is due to the different masses causing different velocities. Each ion mass will thus have a different flight-time to the detector. Other ionization techniques include rapid evaporative ionization mass spectrometry (REIMS) and desorption electrospray ionization (DESI) mass spectrometry.

MALDI-TOF mass spectroscopy can quantify intact proteins in biological tissue and fluids, and is thus applicable to direct analysis of biological tissues and single cell organisms with the aim of characterizing endogenous peptide and protein constituents. Quantification by MALDI-TOF mass spectrometry may be absolute or relative in nature, and it may require the use of internal standards and/or corrections for matrix effects, ionization efficiency, and suppressive effects. The sample is generally mixed with a matrix material which facilitates desorption and ionization of the sample. The mass-to-charge ratio (m/z) is measured as a function of travel time, where separation of the fragments is due to the different masses causing different velocities through the drift space and different times of flight to the detector.

Analysis of cells or tissue samples using mass spectrometry provides different protein profiles. Analysis of the protein profiles from known samples may be used to generate proteomic signatures for each, which may be dependent on the type of tissue and the type or severity of the disease. These proteomic signatures may then be used to predict or classify an unknown sample. For example, the proteomic profile of an unknown sample may be compared to reference profiles from one or more reference samples to classify the unknown sample as one of a feline inflammatory bowel disease and feline small cell lymphoma. Similarly, this concept may be extended to other types of biomarkers present in the sample, such as lipids, carbohydrates, metabolites, endogenous peptides, and other small molecules.

The reference sample may comprise a sample from a subject having a known clinical outcome, e.g., feline inflammatory bowel disease and feline small cell lymphoma. The reference sample may comprise a sample from a subject having an outcome established by multiple concordant diagnoses by pathologists, e.g., having 2, 3, or more pathologists concur on the diagnostic state of a subject based on review of existing clinical, histopathological, and/or molecular diagnostics data. The reference sample may comprise a sample from a subject having an outcome established by a review panel of medical or veterinary specialists, e.g., having 2, 3, or more pathologists, internal medicine specialists, oncologists, etc., concurring on the diagnostic state of a subject based the existing clinical, histopathological, and/or molecular diagnostics data. The reference sample may be used to generate the reference profile. For example, the reference profile may be generated from one or more reference samples, such as at least 5, 10, 25, 50, 100, or more reference samples.

Subjecting the sample from the subject to mass spectrometry may comprise ionizing at least a portion of the sample from the patient using laser energy to generate one or more ions. Obtaining a mass spectrometric proteomic profile from the sample may comprise experimentally measuring the mass to charge ratios of ions to obtain an experimental value corresponding to the mass to charge ratio of each of the ions and distinguishing candidate ions having substantially the same mass to charge ratio. The method may comprise experimentally measuring the mass to charge ratios of the ions using a quadrupole mass analyzer or a Time of Flight mass analyzer.

For example, the method may comprise conducting MALDI-TOF mass spectrometry on a set of tissue samples from a plurality of patients having feline inflammatory bowel disease and/or feline small cell lymphoma; storing a development set including at least one member having feature values of mass spectrometry data generated as a result of conducting MALDI-TOF mass spectrometry on the set of tissue samples; assigning a classification label to each member of the development set based on whether or not the patient associated with the sample has feline inflammatory bowel disease and/or feline small cell lymphoma; separating the development set into at least one of a reference set and a test set; constructing at least one of a feline inflammatory bowel disease and a feline small cell lymphoma classifier using one or more of the feature values; filtering the training set by operating on the training set with the classifier and retaining only those members of the training set that meet a performance threshold of classification accuracy; and evaluating the performance of the classifier on the test set. The method may comprise evaluating the performance of the classifier on the reference set including mass spectrometry data of healthy patients and retaining only those members of the reference set having a classification accuracy that meet performance threshold of classification accuracy. The classifier may comprise at least one feature, such as a single feature, a pair of features, or triplets of features in the set of feature values. The performance threshold of classification accuracy may comprise at least 80%, at least 85%, at least 90%, at least 95%, at least 97%, and at least 99%. The at least one feature may comprise a peptide peak.

The mass spectrometric profile may comprise one or more peptide peaks at one or more of m/z 610.3883±0.1; m/z 627.4335±0.1; m/z 659.4198±0.1; m/z 678.3355±0.1; m/z 733.4526±0.1; m/z 802.448±0.1; m/z 943.6057±0.1; m/z 971.5833±0.1; m/z 1068.6376±0.1; m/z 1082.629±0.1; m/z 1093.5984±0.1; m/z 1096.6205±0.1; m/z 1105.553±0.1; m/z 1243.6573±0.1; m/z 1270.76225±0.1; m/z 1402.73355±0.1; m/z 1469.73305±0.1; and m/z 1953.9388±0.1.

The present invention provides improved methods for differentiating between feline inflammatory bowel disease and feline small cell lymphoma. More specifically, the present invention includes a targeted approach in which only discrete areas within a tissue sample are analyzed. Histological staining may also be used to guide the acquisition of mass spectrometry images so that each area analyzed may be enriched for a single cell type. Histological staining may include hematoxylin and eosin, immunohistochemistry staining, fluorescent staining, and/or other standard histological staining techniques. Such analysis may be more conducive to statistical analysis and classification algorithm generation. Further, such methods may provide a biological insight into the classification which is not attainable through standard histological techniques (i.e., disease outcome, improved diagnostics, treatment responses, etc.). Digital pathology may also be used to guide the acquisition of mass spectrometry images so that each area analyzed may be enriched for a single cell type. Statistical correlation models were generated using a machine-learning algorithm, wherein the algorithm comprises one of a machine learning algorithm including: Genetic algorithm, Support vector machine, Linear discriminant analysis, Random Forest, Bayesian classifier, Rule-based learners, Decision trees, Artificial neural networks, K nearest neighbors, Naïve Bayes.

A genetic algorithm is a machine-learning algorithm that is similar to classical genetics. Initially, peaks from the spectra may be formed into groups (individuals) and evaluated for how well they may differentiate between the phenotypes (benign and malignant). The ones that perform poorly may be discarded while the best may be bred to form a new generation of offspring. The offspring may be then evaluated for their robustness and the best move on to the next generation. Additionally, mutations may be introduced to the sets of peaks at a user-defined rate to increase “genetic” variability. A maximum number of generations is set that the genetic algorithm is allowed to run over, although, this is often not reached as the calculations are terminated when a local optimum is obtained.

A statistical software application such as R code may be used to generate an optimized peak list for distinguishing between disease states. Specialized codes (e.g., R Studio) may process spectra to find an optimal combination of peaks that indicate the disease states. The R code statistical application may be used to generate a combination of peaks that differentiates between feline inflammatory bowel disease (IBD) and feline small cell lymphoma (SCL). For example, a training set of 39 samples comprised of 22 IBD samples and 17 SCL samples. A diagnosis of either feline inflammatory bowel disease and feline small cell lymphoma was rendered on a separate validation set of 54 samples, comprised of 24 IBD samples and 30 SCL samples based on the proteomic signature, which diagnosis was then correlated with the histopathologic diagnosis and consensus diagnosis by a review panel of veterinary medicine specialists that included anatomic pathologists, oncologists, and internal medicine specialists. Using the genetic algorithm, mass spectrometry imaging classified 48 cases correctly. The sensitivity for recognizing feline inflammatory bowel disease and feline small cell lymphoma was 87% and the specificity was 92%.

A linear discriminant analysis is a machine-learning algorithm that may be used to dimensionally reduce a dataset onto a lower-dimensional space. In a linear discriminant analysis, the computer program is taught to recognize a subset of peaks from the IBD samples and the SCL samples. The subset of peaks from spectra is used as a fingerprint for a diagnosis. Each new spectrum from a new sample is compared to these fingerprints and matched to the one to which it is more similar. All other peaks and parts of the spectra are ignored. Within HGMS, multiple annotated areas of a tissue sample may be analyzed for unique diagnostic information related to the individual areas of cells, which when combined and analyzed together may help provide a more accurate diagnosis for the sample and patient as a whole.

Thus, mass spectrometry imaging may be used to differentiate between feline inflammatory bowel disease and feline lymphoma in formalin-fixed, paraffin-embedded tissue sections based on proteomic differences. Mass spectrometry imaging may be an objective and reliable method that may be helpful in difficult cases, in which rendering a firm diagnosis of either feline inflammatory bowel disease and feline lymphoma may be very difficult. The identification of protein expression profiles, which discriminate between feline inflammatory bowel disease and feline lymphoma, may lead to the discovery of clinically useful protein biomarkers that may be incorporated into standard diagnostic and treatment strategies.

Annotation of the sample may occur before part of or the entire sample has been subjected to mass spectrometry, such that selection of the areas of interest if performed pre-acquisition of mass spectrometry data. In this example, the sample may be analyzed in its entirety, or it may be analyzed only at one, multiple, or all annotated locations within the sample. An annotated sample image may be used as a guide or training image to teach the mass spectrometer where to perform the mass spectrometry analysis. In one example, a serial section of the sample may be stained, and the digital microscopy image annotated for regions of interest; these regions of interest may be superimposed onto the image of the sample used in the mass spectrometry analysis to isolate the annotated areas of interest for analysis.

Annotation of the sample may occur after part of or the entire sample has been subjected to mass spectrometry such that selection of the areas of interest is performed post-acquisition of mass spectral data. In this example, the analyzed sample may be stained, and the digital microscopy image annotated for regions of interest; these regions of interest can be superimposed onto the image of the sample use in the mass spectrometry analysis to isolate their components of the spectral data file. In another example, a serial section of the sample may be stained, and the digital microscopy image annotated for regions of interest; these regions of interest can be superimposed onto the image of the sample used in the mass spectrometry analysis to isolate their components of the spectral data file. In another example, the sample may be annotated using the same sample that was subjected to mass spectrometry and staining that same sample for conversion to a digital image for annotation.

The method may comprise collecting a first serial section of the sample and a second serial section of the sample, staining the first serial section of the sample, and subjecting the second serial section of the sample to mass spectrometry. For example, a first serial section of the sample may be stained and a second serial section may be subjected to mass spectrometry. In other words, the treated sample may not be subjected to mass spectrometry. Instead, the regions of the other one of the serial sections that correspond to the annotated sections of the treated sample may be subjected to mass spectrometry.

A method of differentiating feline inflammatory bowel disease and feline lymphoma may generally comprise conducting MALDI-TOF mass spectrometry on the tissue sample with a mass spectrometer; generating a test profile comprising a plurality of mass-to-charge (m/z) intensity values from the tissue sample in a spectrum produced by the mass spectrometer; comparing the test profile and reference profiles (i.e., the IBD reference profile and/or SCL reference profile) obtained from a plurality of other tissue samples having at least one of feline inflammatory bowel disease and feline small cell lymphoma; and classifying the tissue sample as one of feline inflammatory bowel disease and feline small cell lymphoma based on a level of similarity between the test profile of the tissue sample and the reference profiles, wherein the reference profiles include a statistical average profile from a plurality of known normal tissue samples, known feline inflammatory bowel disease tissue samples, and/or known feline small cell lymphoma tissue samples.

The method may comprise treating the sample prior to and/or after subjecting the sample to mass spectrometry. Treating a sample may comprise at least one of collecting at least one serial section of the sample, staining the sample, subjecting the sample to deparaffinization and antigen retrieval, subjecting the sample to on-tissue tryptic digestion, applying a MALDI compatible matrix to the sample, annotating a digital microscopy image of the reference profile and imposing the annotated digital microscopy image of the reference profile onto an image of the sample. For example, the method may comprise subjecting the sample to deparaffinization, subjecting the sample to antigen retrieval, subjecting the sample to on-tissue tryptic digestion, and/or applying a MALDI compatible matrix to the sample prior to subjecting the sample to mass spectrometry. For example, the method may comprise staining the sample prior to subjecting the stained sample to mass spectrometry. In another example, the method may comprise staining the sample after subjecting the sample to mass spectrometry. Annotating the sample may comprise marking targets, or areas of interest on the sample that may have a diameter from 10-500 micrometers, such as 50 micrometers and 250 micrometers. Subjecting the sample to mass spectrometry may comprise subjecting the targets or areas of interest to mass spectrometry. Subjecting the sample to mass spectrometry may comprise subjecting the entire sample, i.e., targets or areas of interest as well as other areas of the sample, to mass spectrometry.

The method may have a sensitivity and a specificity of at least 75% in correctly classifying the tissue sample, such as at least 80%, at least 85%, at least 90%, at least 95%, or even at least 99%.

The method may comprise repeating one or more of the steps described above on the tissue sample, such as, for example, in 6-12 months, 6-18 months, 6-24 months, 12-18 months, 12-24 months, 18-24 months, or longer when the patient is identified as lacking feline inflammatory bowel disease and feline lymphoma.

The classification may comprise use of a genetic algorithm or a linear discriminant analysis to generate a proteomic signature of a reference sample. The reference sample may include a proteomic signature of a known feline inflammatory bowel disease and known feline lymphoma. The known feline lymphoma may comprise small cell lymphoma, intermediate cell lymphoma, large cell lymphoma, or large granular lymphocyte lymphoma. The mass spectrometric profile may comprise analysis of a specific subset of markers as defined by the genetic algorithm or by an R code statistical program. The mass spectrometric profile may comprise an average spectrum from a known normal tissue, known feline inflammatory bowel disease and known feline lymphoma. The mass spectrometric profile may comprise an average spectrum from known feline inflammatory bowel disease and known feline lymphoma.

The mass spectrometry may comprise secondary ion mass spectrometry, laser desorption mass spectrometry, matrix assisted laser desorption mass spectrometry, electrospray mass spectrometry, desorption electrospray ionization, or laser ablation electrospray ionization mass spectrometry.

The method may comprise generating the sample from the patient.

The method may comprise making a treatment decision for the patient.

The method may comprise treating the patient with at least one of chemotherapy, immunotherapy, toxin therapy, surgery, and radiotherapy when the patient is identified as having feline inflammatory bowel disease and feline small cell lymphoma.

The method may comprise assessing one or more patient history parameters from the patient.

The method may comprise performing histologic analysis on the sample.

The method may comprise performing a mass spectrometric analysis of a tissue sample having one of known normal tissue, known feline inflammatory bowel disease, and known feline lymphoma.

The method may comprise making a prediction of the patient's survival based on the classification.

The method may comprise determining a form of treatment, e.g., selecting a drug, based on the classification.

The statistical average profile of the method may be generated using a genetic algorithm which defines a mass spectrometric profile comprising one or more markers.

In various embodiments, the mass spectrometric profile may comprise one or more markers. In various embodiments, the mass spectrometric profile may comprise one or more peptide peaks at one or more of m/z 600-4000. In various embodiments, the mass spectrometric profile may comprise one or more peptide or protein peaks at one or more of m/z 600-25,000. For example, the profile may comprise at least one of m/z 610.3883±0.1; m/z 627.4335±0.1; m/z 659.4198±0.1; m/z 678.3355±0.1; m/z 733.4526±0.1; m/z 802.448±0.1; m/z 943.6057±0.1; m/z 971.5833±0.1; m/z 1068.6376±0.1; m/z 1082.629±0.1; m/z 1093.5984±0.1; m/z 1096.6205±0.1; m/z 1105.553±0.1; m/z 1243.6573±0.1; m/z 1270.76225±0.1; m/z 1402.73355±0.1; m/z 1469.73305±0.1; and m/z 1953.9388±0.1.

The method may comprise using the mass spectrometric profile to determine which form of treatment may be more therapeutically effective for the patient. In various embodiments, the form of treatment may comprise a drug compound dosage regimen, antibody-drug conjugate dosage regimen, radiochemical compound dosage regimen, radiation therapy, and/or surgical excision,

The method may comprise immunohistochemical analysis on the tissue sample. In various embodiments, the patient may previously have had immunohistochemical analysis of the tissue sample. In various embodiments, the previous immunohistochemical analysis indicated that the tissue sample was one of normal tissue, feline inflammatory bowel disease and feline lymphoma. In various embodiments, the previous immunohistochemical analysis indicated that the tissue sample was one of feline inflammatory bowel disease and feline lymphoma.

The statistical average profile of the method may be generated using a linear discriminant analysis on all or a subset of peaks from the total spectrum from the plurality of known normal tissue, the plurality of known feline inflammatory bowel disease and the plurality of known feline lymphoma. The linear discriminant analysis may show a sensitivity and a specificity of at least 75% in correctly classifying the tissue sample. The linear discriminant analysis may also show a sensitivity and a specificity of at least 80%, 85%, 90%, 95% or greater in correctly classifying the tissue sample

The plurality of known normal tissue, the plurality of known feline inflammatory bowel disease, and the plurality of known feline lymphoma may be classified by immunohistochemical analysis, genetic analysis, patient clinical outcome, or a combination thereof.

Without wishing to be bound to any particular theory, subjecting the sample to mass spectrometry prior to annotation may reduce the reproducibility of results for one or more of the following reasons: exposure to the laser may cause mild to moderate destruction of the sample which may impede proper observation and analysis of the sample for staining and annotation; the use of larger regions of the sample for analysis may encourage less particularity when defining the region for cancer cell analysis; and decreased particularity may result in a wider array of cell types within the sample increasing the variability of results. Limiting the data to the areas of interest may provide for more accurate classification and results as there is less “noise” from inappropriate cell types. Noise, in this context, consists of signals originating from other cell types than the ones of interest. This may include red blood cell, inflammatory cells, and the like.

The mass spectrometric proteomic profile may include one or more peaks of monoisotopic mass or peaks arising from one or more isotopes of the biomarkers. The intensity of or area under the peak for the monoisotopic or isotopic mass may be used to determine the abundance value. The monoisotopic mass is the sum of the masses of the atoms in a molecule using the unbound, ground-state, rest mass of the principal (most abundant) isotope for each element instead of the isotopic average mass. Monoisotopic mass is typically expressed in unified atomic mass units (u), also called Daltons (Da). An isotopic mass is the sum of the masses of the atoms in a molecule using the ground-state, rest mass of each isotope that comprises a molecule. For example, for biomolecules, it is common to have detectable isotopes that have mass shifts due to the presence of carbon-13 and its natural abundance of 1.1%. The mass spectrometric proteomic profile may not include all of the peaks detected from the sample. Without wishing to be bound by theory, it is thought that the peaks represent unique biomarkers that are useful for fast detection and identification of cancer in the samples or biomarkers or that are useful for determining prognosis for a patient. These biomarkers do not need to be identified (e.g., myosin) to be used as mass signature components of the mass spectrometric proteomic profile. When samples that have been enzymatically digested as part of the treatment process, more than one peak may be associated with the same biomarker as a result of the fragmentation of the protein parent molecules into constituent sub-protein level biomolecules (e.g., peptides, glycans). For example, one protein biomarker may have several peptides detected and comprising part of the mass spectrometric proteomic profile.

As discussed above, a diagnosis of feline inflammatory bowel disease and feline lymphoma may be based on characteristic similarities or differences (e.g., proteomic signature) between tissue samples and/or a tissue sample and reference sample. For example, if the proteomic profile is presented in the form of a mass spectrum, the proteomic signature may be a peak or a combination of peaks that differ, qualitatively or quantitatively, from the mass spectrum of a corresponding reference sample. Thus, the appearance of a new peak or a combination of new peaks in the mass spectrum, or any statistically significant change in the amplitude or shape of an existing peak or combination of existing peaks, or the disappearance of an existing peak in the mass spectrum may be considered a proteomic signature for one of feline inflammatory bowel disease and feline lymphoma.

Statistical methods for comparing proteomic profiles may be defined by the peak amplitude values at key mass/charge (m/z) positions along the horizontal axis of the mass spectrum. A characteristic proteomic profile may, for example, be characterized by the pattern formed by the combination of spectral amplitudes at given m/z vales. The presence or absence of a proteomic signature for one of a feline inflammatory bowel disease and feline lymphoma, or the substantial identity of two proteomic profiles, may be determined by matching the proteomic profile of a tissue sample with the proteomic profile of a reference sample using an appropriate algorithm.

The presently disclosed invention provides improved methods for differentiating between feline inflammatory bowel disease and feline lymphoma. More specifically, the present invention includes a targeted approach in which only discrete areas within a tissue sample are analyzed. Histological or immunohistological staining may also be used to guide the acquisition of mass spectrometry imaging so that each area analyzed may be enriched for a single cell type. Such analysis may be more conducive to statistical analysis and classification algorithm generation. Further, such methods may provide a biological insight into the classification which is not attainable through standard histological techniques (i.e., disease outcome, improved diagnostics, treatment responses, etc.). The present invention may include samples representing multiple subtypes of feline inflammatory bowel disease and feline lymphoma, as well as samples representing both the training and validation sets that have been collectively shown to provide an increase in the overall accuracy of a classification algorithm and model and increase in its utility for evaluating samples of unknown subtype.

According to the present invention, a tissue sample is collected onto an indium tin oxide (ITO) coated glass slide that is compatible with a MALDI TOF mass spectrometer. A serial section is collected onto a standard microscopy slide and stained with hematoxylin and eosin (H&E). The stained section is scanned using a digital slide scanner and made available to a pathologist who annotates histological regions of interest within the section. The annotated stained section is registered with a digital image of the serial unstained section using Photoshop® (or equivalent software) allowing for the coordinates of the annotations to be obtained. The unstained section is subjected to sample preparation including deparaffinization, antigen retrieval, tryptic digestion, and matrix application. Mass spectra are collected from the designated locations and the spectra subjected to statistical analysis.

A biomarker in the tissue sample may be modified before analysis to improve its resolution or to determine its identity. For example, the biomarkers may be subject to proteolytic digestion before analysis using a protease. Proteases, such as trypsin, PNGaseF, and GluC, that are likely to cleave the biomarkers into a discrete number of fragments may be particularly useful. Similarly, heat-induced digestion and chemical cleavage can generate reproducible peptide fragments from proteins. Due to their individual characteristics, each fragment will be detected as a unique mass and will experience differences in ionization efficiency, resolution, and sensitivity during mass spectrometry analysis. One, multiple, or all fragments of a biomarker may be detected in a single analysis. The fragments that result from digestion function as a fingerprint for the biomarkers, thereby enabling their detection indirectly. This may be particularly useful where there are biomarkers with similar molecular masses that might be confused for the biomarker in question. Also, proteolytic fragmentation may be useful for high molecular weight biomarkers because smaller biomarkers may be more easily resolved by mass spectrometry. Typically, the peptide fragments resulting from the enzymatic digestion of a protein biomarker are more easily detected than the original parent protein itself due to improved ionization efficiency and desorption efficiency, leading to improved sensitivity. Additionally, proteolytic digestion allows for measurement of uncrosslinked protein segments from larger proteins that have been crosslinked due to fixation processes. This is particularly useful in FFPE tissues where large protein biomarkers are crosslinked and very difficult to liberate from the tissue surface through desorption ionization processes. Carefully controlled enzymatic digestion treatment that maintains spatial localization increases the sensitivity of detection for the resultant biomarker fragment peptides, glycans, in a robust and reproducible manner. In a histology guided mass spectrometry approach, this sample treatment process results in complex biomarker fragment spectra for each type of cell subpopulation, for which these types of large, robust data sets are well-suited for statistical analysis by machine learning and artificial intelligence platforms. In another example, biomarkers may be modified to improve detection resolution. For instance, neuraminidase may be used to remove terminal sialic acid residues from glycoproteins to improve binding to an anionic adsorbent and to improve detection resolution. In another example, the biomarkers may be modified by the attachment of a tag of particular molecular weight that specifically binds to molecular biomarkers, further distinguishing them.

### EXAMPLES

The present invention may be better understood when read in conjunction with the following representative examples. The following examples are included for purposes of illustration and not limitation.

Twenty cats were included in the study. Clinically healthy, adult, client-owned cats 3 years of age, undergoing an elective procedure requiring general anesthesia were eligible for enrollment in the study. Study eligibility initially was determined by an owner questionnaire on general and gastrointestinal health. The questionnaire covered the following areas: attitude, activity, appetite, drinking, urination, chronic illnesses, weight loss, vomiting, diarrhea, and treatment with antibiotics, antacids, anti-inflammatory drugs, or corticosteroids. A physical examination was performed by a single board-certified internist (SM). Blood was collected from a peripheral vein or the jugular vein and the following tests were performed: complete blood count (CBC), serum biochemistry profile, serum total T4 concentration, and serum concentrations of cobalamin, folate, feline pancreatic lipase immunoreactivity (fPLI), and feline trypsin-like immunoreactivity (fTLI). Additionally, a serum feline immunodeficiency virus antibody test and feline leukemia virus antigen test were performed if the status of the cat was unknown (n=4). Cats with gastrointestinal signs (e.g., weight loss, hyporexia, vomiting more than twice per month, and diarrhea) within 6 months before enrollment were excluded. In addition, cats with systemic diseases, chronic illnesses, or laboratory abnormalities that were deemed to be clinically relevant were excluded from the study. Cats with a serum cobalamin concentration less than 350 ng/L also were ineligible. Finally, cats that had received any antibiotics, antacids, anti-inflammatory drugs, or corticosteroids within the past 6 months were excluded from the study.

After a routine dental procedure under general anesthesia, all cats underwent gastroduodenoscopy. Six biopsy specimens each from the stomach and upper small intestine (SI) tract were collected for histopathologic examination, immunohistochemistry, and clonality testing. In 1 cat, anesthesia time and personnel allowed for an additional ileo-colonoscopy, whereas in 2 cats only duodenal biopsy specimens were collected because of a longer than usual setup time.

Histopathologic examination of hematoxylin and eosin (H&E)-stained endoscopic formalin fixed paraffin embedded (FFPE) tissue sections was performed by a single board-certified pathologist (MA) blinded to the clinical status of the cats (i.e., the pathologist was unaware that the samples were from healthy control cats). Findings were reported descriptively, and numerically scored according to the World Small Animal Veterinary Association (WSAVA) histopathologic scoring system. Briefly, morphological features (e.g., surface epithelial injury, crypt hyperplasia, crypt dilatation or distortion, and fibrosis or atrophy) and inflammatory changes (e.g., lamina propria lymphocytes, plasma cells, eosinophils, neutrophils, and macrophages) were assessed histologically and assigned a score (normal=0, mild=1, moderate=2, and marked=3).

Sections of FFPE tissue were sent to a single external laboratory for immunohistochemistry and clonality testing (i.e., the immunohistochemistry and molecular clonality analyses of biopsy specimens were performed at the Leukocyte Antigen Biology Laboratory, University of California Davis, SVM-PMI, 1 Shields Ave, 4206 VM3A, Davis, Calif. 95616, on a fee-for-service basis). Pathologists at the external laboratory were blinded to the health status of the cats. Biopsy sections were reassessed with H&E staining, and then immunohistochemistry and clonality testing were performed.

Immunohistochemistry was conducted using a stepwise approach. Staining for T-, B-, and natural killer (NK) cell markers (e.g., CD3, CD79a, and granzyme B, respectively) were performed at the external pathologist's discretion and based on the results of the H&E staining (e.g., size and distribution of mucosal lymphocytes).

Molecular clonality testing was conducted on at least 2 sections (each 25 μm) of FFPE tissue using PARR analysis. Total DNA content was measured before the PCR procedure to ensure that enough tissue was present for accurate PARR testing. Results from the H&E-based histopathology, immunohistochemistry, and molecular clonality analysis were integrated and reported by the external pathologist.

All cats were followed up after endoscopy at various time points. An owner questionnaire on general and gastrointestinal health since endoscopy was used to assess the cat's health status. When abnormal findings were reported, owners were contacted and a more detailed history was obtained.

The association between the results of laboratory tests, histopathology, and clonality assays was assessed using chi-squared tests or a Fisher's exact test, as appropriate. Statistical significance was set at P<0.05. Statistical analyses were performed using a statistical software package (GraphPad Prism, GraphPad Software, Inc, San Diego, Calif.).

Twenty cats were included in the study. Cats had a median age of 9.5 years (range, 3-18 years), median body weight of 5.0 kg (range, 2.6-10.8 kg), and median body condition score of 6 out of 9 (range, 5-9). There were 12 female spayed and 8 male neutered cats. Breeds included domestic shorthair (n=12), domestic longhair (n=3), Siamese (n=2), Burmese (n=1), Norwegian Forest Cat (n=1), and Persian (n=1).

According to the owner, 1 cat had a short episode of acute self limiting diarrhea within the 6 months before the study. This cat had a minimally increased serum folate concentration (22.4 μg/L; reference interval, 9.7-21.6 μg/L). One cat had an increased fPLI concentration (15.6 μg/L; reference interval, 3.5 μg/L) without any current or prior associated clinical signs. Five cats had increased serum folate concentrations (25.3, 27.3, 33.8, 62.5, and 65.5 μg/L) without any current or prior associated clinical signs.

Gastric biopsy specimens were available from 18 cats, and upper intestinal tract biopsy specimens were available from all 20 cats. Demographic characteristics, histopathological findings, and results of molecular clonality testing are shown in Table 1. Detailed results for individual cats are shown in Supporting Information Table 51.

Sample number and quality were reported by the pathologist to be adequate for all cats. A detailed list of the WSAVA scores is shown in Supporting Information Table S2.

Histopathologic evaluation of H&E-stained gastric biopsy sections had abnormalities in each of the 18 available cats. Lymphocytic-plasmacytic gastritis was identified in all cats. This finding was reported to be minimal in 4 cats, minimal to mild in 2 cats, mild in 5 cats, mild to moderate in 5 cats, and moderate in 2 cats. One of the 2 cats with moderate lymphocyticplasmacytic gastritis was reported to have focally extensive nodular lymphocytic and plasmacytic gastritis. Fibrosis was the most commonly reported morphologic abnormality, present in 17 cats (minimal in 3 cats, minimal to mild in 2 cats, mild in 11 cats, and moderate in 1 cat); lymphocytic nodule formation was present in 2 cats and 1 cat had occasional mucosal cyst formation.

Histopathologic evaluation of duodenal biopsy specimens showed some degree of lymphocytic-plasmacytic mucosal infiltration in all 20 cats (minimal to mild in 4 cats, mild in 4 cats, mild to moderate in 6 cats, and moderate in 4 cats). In 2 cats, a diagnosis of ISCL was made based on histopathology. In addition, to a diffuse infiltration of the lamina propria with monomorphic small lymphocytes, both cats had moderate epithelial infiltration with small lymphocytes. Morphologic changes were present in 19 cats. The most common architectural change observed in the duodenum was crypt hyperplasia in 18 cats (minimal in 3, minimal to mild in 1, mild in 6, and mild to moderate in 7), followed by fibrosis in 4, and lacteal dilatation in 4. One cat was reported to have occasional crypt abscesses.

Lymphocytes infiltrating the lamina propria stained positive for CD3 in all cats. In 12 cats, a CD3+ epitheliotropic lymphocyte population was reported. In 5 cats, a mixed epitheliotropic and lamina proprial lymphocytic infiltrate staining positive for CD3 was identified. In 3 cats, the pathologist reported a CD3+ lymphocytic infiltrate without further comments on localization. In no case did the pathologist suggest that any additional stains were needed for a final diagnosis.

Molecular clonality testing of T cell receptor genes (TRG) was performed in all cats. In addition, clonality analysis using B-cell primers including IgH2, IgH3, and K-deleting element was performed on sections of duodenal biopsy specimens from 1 cat.

Molecular clonality testing of the TRG identified clonal rearrangements in duodenal biopsy specimens of 8 cats. In 5 cats, clonal rearrangements in a polyclonal background were reported. In 6 cats, including the single case in which T- and B-cell primers were used, rearrangements were determined to be polyclonal. In 1 cat, TRG clonality analysis was interpreted as pseudoclonal, likely because of insufficient DNA retrieval.

Results from H&E stains, immunohistochemistry, and PARR were integrated by the external pathologists and reported as case interpretations. Results were interpreted as consistent with duodenal ISCL in 12 cats and emerging ISCL in 1 cat. Six cats were reported as having lymphocytic enteritis. In 1 cat, the findings were deemed uninterpretable because of pseudoclonality.

No association was found between laboratory findings and histopathology or results of clonality testing (P=0.28 and 0.18, respectively). Similarly, no association was identified between histopathology and results of clonality testing (P=0.38).

Follow-up data were available for all 20 cats. Two cats were euthanized because of signs of gastrointestinal disease, including weight loss and vomiting, 295 and 654 days post-endoscopy, respectively (see Supporting Information Table 51, cases 10 and 19). Both cats were diagnosed previously with ISCL based on the first histopathologic examination as well as laboratory results on H&E, immunohistochemistry, and PARR. The owner reported that the former cat had developed weight loss (about 1 kg) and frequent vomiting about 9 months after endoscopy. The cat underwent abdominal ultrasound examination before euthanasia during which thickened segments within the SI tract and abdominal lymphadenomegaly were identified. This cat did not receive any treatment. The second cat developed weight loss (1.3 kg), sarcopenia, vomiting, and chronic kidney disease (International Renal Interest Society stage 3, proteinuric, non-hypertensive). The cat was treated with prednisolone and budesonide before euthanasia eventually was elected. Neither cat was available for postmortem examination.

Another cat developed severe non-self-limiting vomiting approximately 513 days post-endoscopy (see Supporting Information Table 51, case 13). The owner reported that a CBC, serum biochemistry profile, and abdominal ultrasound examination had been within normal limits and that the cat's body weight had been unchanged after endoscopy. Upon treatment with a prescription hydrolyzed protein diet, signs of gastrointestinal disease ceased within days. At the time of study enrollment, this was the only cat in which ileo-colonoscopy was performed in addition to gastroduodenoscopy. Histopathology at that time showed mild diffuse and moderate nodular lymphocytic-plasmacytic gastritis with mild fibrosis, mild diffuse lymphocytic-plasmacytic duodenitis with minimal diffuse hyperplasia of crypts, minimal diffuse lymphocytic-plasmacytic ileitis, and mild diffuse lymphocytic-plasmacytic colitis with multifocal nodular lymphocytic aggregates. The WSAVA scores of the stomach, duodenum, and colon were 2.5, 2.0, and 2.0, respectively. Immunohistochemistry was consistent with a mildly epitheliotropic lymphocyte population staining positive for CD3 in both the upper and lower SI tract. Clonality testing identified polyclonal rearrangements in samples from the upper and lower SI tract, with small reproducible peaks within the polyclonal background, suggestive of a decreased T cell receptor repertoire. Lesions were interpreted as consistent with lymphocytic enteritis with mild epitheliotropism in the upper SI tract and lymphocytic enteritis within the lower SI tract.

Owners' responses to follow-up questionnaires for the remaining 17 cats indicated no signs of chronic gastrointestinal disease after a median of 709 days post-endoscopy (range, 219-869 days). Of these 17 cats, duodenal biopsy specimens previously had been interpreted as consistent with ISCL in 10 cats, emerging ISCL in 1 cat, and lymphocytic enteritis in 5 cats. One case was deemed uninterpretable because of pseudoclonality.

Without wishing to be bound to any particular theory, it is believed that present invention is the first study describing the results of histopathology, immunohistochemistry, and molecular clonality testing in endoscopically obtained upper SI biopsy specimens of clinically healthy client-owned cats with demographic characteristics similar to those of cats that present with signs of chronic enteropathy (CE).

All 20 cats in this study had histopathological changes that were considered abnormal based on current WSAVA standards. Although many inflammatory changes were considered minimal to mild, most of the cats had inflammatory lesions that were rated as mild to moderate lymphocytic-plasmacytic enteritis. Two cats were diagnosed with ISCL based on histopathology alone. Other histological features seen in this population of clinically healthy cats included lymphocytic nodule formation in the stomach, fibrosis, crypt hyperplasia, lacteal dilatation, and crypt abscesses.

Results of histopathologic studies of intestinal biopsy specimens from clinically healthy cats have been described before and are the basis for the WSAVA histopathological standards for the diagnosis of gastrointestinal inflammation in endoscopic biopsy specimens from both dogs and cats. However, upon careful examination of the reference population for the WSAVA standards, the definition of a normal histological baseline becomes somewhat questionable. Although guidelines for establishing a normal histopathological baseline may be lacking, guidelines exist for establishing reference intervals for laboratory values. Generally, a reference interval is defined as an interval that, when applied to the population serviced by the laboratory, correctly includes most of the subjects with characteristics similar to the reference group, but excludes others. In other words, a reference may be established in a cohort of healthy individuals with otherwise the same characteristics as the patient population to which it is intended to be compared. The same principle may apply to establishing histopathologic standards. Depending on the study and underlying diagnosis of IBD or ISCL, cats with CE have a reported median age from 6-13.5 years. However, the WSAVA criteria were developed based mainly on full-thickness biopsies from SPF colony cats that were relatively young. One study used adult cats of undetermined origin from an Animal Control Center in Japan. However, the authors also stated that, based on the dental status, the cats were considered young adults. In addition, this study mostly contributed to the WSAVA criteria through the description of epithelial globular leukocytes, rather than normal architecture and total mucosal leukocytes.

The intestinal tract and its gut-associated lymphoid tissue may be the largest lymphoid organ in the body, with an extremely high plasticity and compensatory capacity. With increasing age and chronic antigen exposure, gastrointestinal histology may change without necessarily representing a pathological condition. Therefore, most of the histopathologic lesions seen in our population of cats may in fact be normal for older cats.

On the other hand, clinical or subclinical CE is common in geriatric cats. Therefore, histopathological changes seen in this population also might reflect true subclinical disease. At least for the 2 cats diagnosed with ISCL on histopathology, this appears likely, because those cats developed severe clinical signs of CE and eventually were euthanized about 10-22 months post-endoscopy. Another cat developed clinical signs of CE with severe vomiting about 17 months post-endoscopy. However, this cat had only minimal to mild histopathological changes in both the upper and lower SI tract, and the lymphocyte population was determined to be polyclonal on PARR. In addition, clinical signs subsided after treatment with a hypoallergenic diet. Without wishing to be bound to any particular theory, it is believed that the long lag time between endoscopy and development of clinical signs, mild histopathological changes and polyclonality at the time of endoscopy, and response to diet may, in this particular cat, show that the clinical signs may have been caused by a new onset of CE unrelated to previous changes, and the disease may be categorized as food-responsive enteropathy. The remaining 17 of 20 cats had no clinical signs of chronic gastrointestinal disease after a median of 709 days postendoscopy (range, 219-869 days), and thus subclinical disease appears less likely for this group of cats.

Despite standardized scoring criteria, interpretation of histopathological findings may vary substantially among pathologists. In addition, the extent of cellular infiltration in the lamina propria is subjective without using computer-assisted morphometry methods. Therefore, a different pathologist may have assessed and scored the biopsy specimens differently. In addition to histopathological examinations by 1 pathologist, samples were sent to an external laboratory for immunohistochemistry and molecular clonality analysis of FFPE duodenal tissue samples. Pathologists at the external laboratory performed their own H&E-staining evaluation and integrated results from H&E-based histopathology, immunohistochemistry, and PARR to formulate a diagnosis and interpretation of each case as performed routinely on samples from cats submitted to the laboratory for diagnostic purposes. Clonality analysis of the TRG revealed clonal rearrangements in 13 of 20 cats with or without a polyclonal inflammatory background, which may be interpreted as consistent with SCL (n=12) or emerging SCL (n=1). Only 6 cats were found to have polyclonal T cell receptor (TCR) rearrangements. One cat was found to have results consistent with pseudoclonality, which may be due to insufficient DNA retrieval.

Without wishing to be bound to any particular theory, an explanation for the frequent finding of clonal rearrangements may be the presence of malignant but indolent lymphocyte clones. This may be a reasonable explanation for the 2 cats that were diagnosed with SCL on histopathology and developed clinical signs of CE much later. The remaining 11 cats may have had SCL elsewhere in the gastrointestinal tract with trafficking neoplastic lymphocytes causing positive clonality results. Lymphocyte trafficking is a well-known phenomenon, allowing naïve and memory T cells to be recruited to the lamina propria or epithelium of the intestinal tract by a process called lymphocyte homing. Trafficking of neoplastic (clonal) lymphocytes may cause detection of clonal TRG rearrangements in the present lymphocyte population without apparent histopathological changes. However, the long disease free follow-up time for these cats may show that this is less likely.

Another explanation would be the occurrence of benign clones. In some instances, chronic antigenic stimulation may lead to disproportional proliferation of a lymphocytic subpopulation, resulting in true but benign clonal expansion, often in a polyclonal background. Benign clonal expansion has been documented in humans and dogs with infectious and autoimmune diseases, neoplasia, and drug administration. Other causes for the detection of clonality in the absence of neoplasia may include canonical rearrangements of certain γδ T cell clones and nonspecific amplification of sequences other than rearranged TRGs. Benign clonal expansion may be a reasonable explanation, especially for the 5 cats in which clonality analysis identified clonal rearrangements in a polyclonal background. However, no association between extent of inflammation and results of the clonality assay were found. Also, even if such benign clonal expansion were to be the reason for the many cats that were positive for PARR, this would severely hamper the clinical usefulness of PARR.

The presence of pseudoclonal results may be another valid explanation. Pseudoclonal profiles may result from a lack of primers covering the rearranged genes, mutation of primer binding sites (common in somatic hypermutation in B-cells), absence of rearranged T cell receptor gamma chains (NK-cell neoplasms), or insufficient target DNA. In PCR-based clonality assays, the amount of input DNA is standardized and determined mainly by the size and amount of tissue available for DNA retrieval. However, the target DNA is the DNA that is amplified during the PCR (ie, DNA from T cells in TRG clonality assays). With low numbers of lesional T cells, the ratio of target DNA to total DNA decreases and preferential amplification and pseudoclonality may occur despite adequate total DNA concentration and purity. This may be a reason that clonality assays are interpreted in the context of histopathology and immunohistochemistry, and thus may be performed in the same laboratory by the same pathologist. In this study, 1 cat was reported to have pseudoclonal rearrangements, most likely because of low target DNA in the sample, and thus results were deemed uninterpretable. In all other cats, the clonality assays had sufficient input DNA for the assay to be performed and interpretations of integrated results from histopathology, immunohistochemistry, and PARR were reported for these cats. Polymerase chain reaction-based analysis of Ig/TCR rearrangements is widely used in human medicine and is considered to be the gold standard for clonality testing. However, both false negative and false positive results have been a problem, especially in the early years of assay use. This has led to the formation of the EuroClonality (BIOMED-2) consortium and the development of standardized multiplex PCR assays for nearly all Ig/TCR targets in humans. This standardization made it technically feasible to bring this test into a routine diagnostic setting. However, besides the analytical phase, pre- and post-analytical aspects should be considered. Thus, interpretation algorithms have been introduced that take into account peak heights and ratios to define truly clonal rearrangements. Such standardization is currently lacking among veterinary laboratories, and thus differences in primers, laboratory practices, and result interpretation among laboratories might explain our findings.

Since the introduction of clonality assays for the diagnosis of intestinal T cell lymphoma in cats in 2005, several studies have investigated the value of PCR-based clonality assays in the diagnosis of intestinal and extraintestinal T cell lymphoma in cats. Subsequently, clonality assays have become the gold standard for the diagnosis and differentiation of lymphoma in cats. However, similar to the WSAVA criteria, PARR for the molecular diagnosis of intestinal T cell lymphoma in cats was developed based on samples obtained from healthy young (i.e., 12-18 months old) SPF colony cats and thus might not be representative of the target population. Although the sensitivity of PCR-based clonality assays performed on FFPE tissue generally is considered high (>90%), a study in human patients with lymphoproliferative disease identified specificities as low as 54.3% in patients with reactive lesions, even with the use of standardized BIOMED-2 clonality assays. A recent study in cats with CE reclassified cats diagnosed with IBD on the basis of histopathology as having SCL instead, based on their PARR analysis. These results, as well as results from human pathology, imply that reclassification based on clonality results alone may not be justified.

Endoscopy and collection of biopsy specimens were restricted to the upper SI tract and stomach in most of the cats to not severely prolong anesthesia time. Accordingly, some cats had SCL elsewhere inside or outside the intestinal tract, which may explain the number of clonal results in the study. Without wishing to be bound to any particular theory, however, this seems unlikely because most of the cats did not develop any clinical signs of CE within several months to years afterwards. In addition, we cannot entirely exclude the possibility of subclinical disease being present in this population of cats. Most of the cats in our study were slightly overweight with a median body condition score of 6 (range, 5 to 9 out of 9), and thus obesity may be viewed as a clinical abnormality. However, for a number of different diseases in humans, mild obesity has been shown to be associated with longer survival compared to patients with a body condition that is considered ideal. 36 Based on our clinical experience, most healthy geriatric cats are overweight, and restricting the body condition score to an ideal score of 5 out of 9 likely would have introduced a substantial bias into the study population. Our inclusion criteria permitted cats that were vomiting up to twice per month. In addition, some cats had laboratory abnormalities such as increased serum folate concentration or increased serum fPLI concentration without associated clinical signs. Cats that were vomiting either were long-haired cats vomiting predominately hairballs or had occasional vomiting up to twice per month without any other clinical signs. Both cats with ISCL were among the 4 cats that had occasional vomiting, and thus we cannot exclude that this might have been an early sign of gastrointestinal disease.

Progression of IBD to ISCL over months to years has long been suspected, and inflammatory lesions frequently coexist with SCL. Although results of histopathology and clonality testing did not correlate with clinical or laboratory abnormalities, subclinical CE remains a possible reason for the findings in our study. Finally, because of the stepwise approach the pathologists took for immunohistochemistry, tissue biopsy specimens did not routinely undergo staining for B or NK cells. Therefore, mixed infiltrates were likely missed in the tissue biopsy specimens. However, we indented not to interfere with the pathologists' approach and to assess whether this cohort of cats would be identified correctly as clinically healthy based on tests that currently are considered to be the gold standard for the diagnosis of CE in cats.

The results of histopathology, immunohistochemistry, and molecular clonality testing in endoscopically-obtained upper SI biopsy specimens from healthy client-owned cats with demographic characteristics resembling those of cats that present with signs of CE has been described. Intestinal biopsy samples commonly had histopathologic findings considered abnormal based on current WSAVA standards. Similarly, results of clonality testing identified many cats with clonal rearrangements within this group of healthy cats. Without wishing to be bound to any particular theory, it is believed that histological scoring criteria may need to be revised and adapted to a more adequate reference population. Although the sensitivity of molecular clonality testing generally is considered to be high, the results may show that further assessment of the specificity of this diagnostic modality may be desirable. Implementations of the mass spectrometry system may be described within the context of a device configured to perform various steps, methods, and/or functionality in accordance with aspects of the present invention. It is to be appreciated that a mass spectrometry system including a computing device or computer system may be implemented by one or more computing devices. Implementations of the mass spectrometry system may be described in the context of a “device configured to”, wherein the term configured may be taken to mean that the device may implement computer-executable instructions that are executed to perform various steps, methods, and/or functionality in accordance with aspects of the present invention.

The following aspects are disclosed in this application:

Aspect 1. A method of analyzing a feline tissue sample comprising: (a) generating sample ions directly from the feline tissue sample using a MALDI ionization source; (b) receiving the ions into a mass spectrometer; (c) identifying at least one of a feline inflammatory bowel disease related compound and a feline small cell lymphoma related compound in the feline tissue sample from results from the mass spectrometer; (d) comparing the related compound in the feline tissue sample to one or more known feline inflammatory bowel disease profiles and feline small cell lymphoma profiles; and (e) identifying at least one condition related to the feline tissue sample from the comparison of the at least one identified related compound to the one or more known profiles.

Aspect 2. The method of aspect 1, wherein the one or more known profiles comprise at least one of m/z 610.3883±0.1; m/z 627.4335±0.1; m/z 659.4198±0.1; m/z 678.3355±0.1; m/z 733.4526±0.1; m/z 802.448±0.1; m/z 943.6057±0.1; m/z 971.5833±0.1; m/z 1068.6376±0.1; m/z 1082.629±0.1; m/z 1093.5984±0.1; m/z 1096.6205±0.1; m/z 1105.553±0.1; m/z 1243.6573±0.1; m/z 1270.76225±0.1; m/z 1402.73355±0.1; m/z 1469.73305±0.1; and m/z 1953.9388±0.1.

Aspect 3. The method of any of the foregoing aspects, wherein the at least one condition comprises at least one of feline inflammatory bowel disease and feline small cell lymphoma.

Aspect 4. The method of any of the foregoing aspects comprising determining a distribution of the one or more analytes within the feline tissue sample.

Aspect 5. The method of any of the foregoing aspects comprising performing a pathological comparison of the feline tissue sample with one of a plurality of reference standards for feline inflammatory bowel disease and a plurality of reference standards for feline small cell lymphoma.

Aspect 6. The method of any of the foregoing aspects, wherein the reference standards differ from each other with respect to at least one of time in cell development, stage of disease progression, culturing or growth conditions, cell type, tissues type, and disease type.

Aspect 7. The method of any of the foregoing aspects, wherein the pathological comparison includes at least one of immunohistochemistry (IHC), immunofluorescence (IF), hematoxylin and eosin (H&E) staining, clonality testing (e.g., PCR for Antigen Receptor Rearrangements (PARR)), and imaging using one of a light microscope, a fluorescence microscope, a confocal microscope, and an electron microscope.

Aspect 8. The method of any of the foregoing aspects comprising: producing a mass spectral image of the feline tissue sample based on results from the mass spectral analysis; performing a histochemistry analysis technique on the feline tissue sample to thereby stain the sample; producing an optical image of the stained sample; overlaying the mass spectral image with the optical image of the stained sample to produce an overlaid image; and determining a distribution of the one or more analytes in the tissue sample based on an analysis of the overlaid image.

Aspect 9. The method of any of the foregoing aspects comprising detecting cancerous tissue within the tissue sample based on an analysis of the overlaid image.

Aspect 10. The method of any of the foregoing aspects comprising experimentally measuring a mass to charge (m/z) ratio of the sample ions using the mass spectrometer.

Aspect 11. The method of any of the foregoing aspects, wherein the mass spectrometer comprises one of a quadrupole mass spectrometer, a time of flight mass spectrometer, an Orbitrap mass spectrometer, and an ion trap mass spectrometer.

Aspect 12. The method of any of the foregoing aspects comprising ionizing the feline tissue sample using laser energy to generate the sample ions directly from the feline tissue sample.

Aspect 13. The method of any of the foregoing aspects, wherein identifying at least one related compound in the feline tissue sample from results from the mass spectrometer comprises generating a mass spectrometry profile of the feline tissue sample from results from the mass spectrometer.

Aspect 14. The method of any of the foregoing aspects characterized by a sensitivity and a specificity of at least 85% in correctly classifying the feline tissue sample as one of feline inflammatory bowel disease and feline small cell lymphoma.

Aspect 15. The method of any of the foregoing aspects comprising staining the feline tissue sample prior to generating sample ions directly from the feline tissue sample using a MALDI ionization source.

Aspect 16. The method of any of the foregoing aspects comprising staining the feline tissue sample after generating sample ions directly from the feline tissue sample using a MALDI ionization source.

Aspect 17. The method of any of the foregoing aspects comprising staining the feline tissue sample with hematoxylin and eosin.

Aspect 18. The method of any of the foregoing aspects comprising: treating the feline tissue sample to deparaffinization and antigen retrieval prior to on-tissue tryptic digestion and matrix application using a robotic sprayer; treating the feline tissue sample to on-tissue tryptic digestion; and applying a MALDI compatible matrix to the treated feline tissue sample using a robotic sprayer.

Aspect 19. The method of any of the foregoing aspects, wherein the feline tissue sample comprises a first section and a second section, and wherein the method comprising: annotating an image of the first section to identify areas of interest on the feline tissue sample having a diameter from 10-400 micrometers; and imposing the annotated image of the first section upon the second section prior to generating sample ions directly from the areas of interest identified on the second section of the feline tissue sample using the MALDI ionization source.

Aspect 20. The method of any of the foregoing aspects, wherein the areas of interest are identified as one of feline inflammatory bowel disease and feline small cell lymphoma using histologic analysis and/or immunohistochemical analysis.

Aspect 21. The method of any of the foregoing aspects comprising determining a treatment for one of feline inflammatory bowel disease and feline small cell lymphoma.

Aspect 22. A method for analyzing a tissue section, wherein the tissue section comprises at least one analyte indicative of feline small cell lymphoma and a plurality of reference analytes that are not associated with the feline small cell lymphoma, the method comprising: conducting a MALDI-TOF technique on one or more locations of the tissue section to ionize the at least analyte and the plurality of reference analytes from the one or more locations of the tissue section and direct the at least one analyte and the plurality of reference analytes into a mass spectrometer to thereby generate a mass spectral signal of the at least one analyte and the plurality of reference analytes mass spectral signals at the one or more locations; normalizing the mass spectral signal of the at least one analyte at the one or more locations to a combination of the plurality of reference analyte mass spectral signals from the tissue section in order to obtain a relative abundance of the at least one analyte at the one or more locations within the tissue section; and producing a mass spectral image of the tissue section that comprises a relative abundance of the at least one analyte at the one or more locations within the tissue section, wherein the relative abundance of the at least one analyte at the one or more locations within the tissue section is indicative of locations of cancerous tissue or non-neoplastic tissue within the tissue section.

Aspect 23. The method of any of the foregoing aspects comprising performing a histochemistry analysis technique on the tissue sample.

Aspect 24. The method of any of the foregoing aspects, wherein the histochemistry analysis technique is H&E staining or immunohistochemistry.

Aspect 25. The method of any of the foregoing aspects comprising producing an optical image of the tissue section based on data from the histochemistry analysis technique.

Aspect 26. The method of any of the foregoing aspects comprising overlaying the mass spectral image of the tissue section onto the optical image of the tissue section.

Aspect 27. The method of any of the foregoing aspects, wherein the mass spectral signals comprise at least one of m/z 610.3883±0.1; m/z 627.4335±0.1; m/z 659.4198±0.1; m/z 678.3355±0.1; m/z 733.4526±0.1; m/z 802.448±0.1; m/z 943.6057±0.1; m/z 971.5833±0.1; m/z 1068.6376±0.1; m/z 1082.629±0.1; m/z 1093.5984±0.1; m/z 1096.6205±0.1; m/z 1105.553±0.1; m/z 1243.6573±0.1; m/z 1270.76225±0.1; m/z 1402.73355±0.1; m/z 1469.73305±0.1; and m/z 1953.9388±0.1.

Aspect 28. The method of any of the foregoing aspects, wherein the method has a sensitivity and a specificity of at least 85% in correctly classifying the feline tissue sample as one of feline inflammatory bowel disease and feline small cell lymphoma.

Aspect 29. The method of any of the foregoing aspects, wherein performing a histochemistry analysis technique on the tissue sample is performed before conducting the MALDI-TOF technique on one or more locations of the tissue section.

Aspect 30. The method of any of the foregoing aspects, wherein performing a histochemistry analysis technique on the tissue sample is performed after conducting the MALDI-TOF technique on one or more locations of the tissue section.

In general, a computer system or computing device may include one or more processors and storage devices (e.g., memory and disk drives) as well as various input devices, output devices, communication interfaces, and/or other types of devices. A computer system or computing device can also include a combination of hardware and software. It should be appreciated that various types of computer-readable storage media can be part of a computer system or computing device. As used herein, the terms “memory”, “computer-readable storage media” and “computer-readable storage medium” do not mean and unequivocally exclude a propagated signal, a modulated data signal, a carrier wave, or any other type of transitory computer-readable medium. The mass spectrometry system may include a processor configured to execute computer-executable instructions and a computer-readable storage medium (e.g., memory and/or additional hardware storage) storing computer-executable instructions configured to perform various steps, methods, and/or functionality in accordance with aspects of the present invention.

Computer-executable instructions may be embodied and/or implemented in various ways such as by a computer program (e.g., client program and/or server program), a software application (e.g., client application and/or server application), software code, application code, source code, executable files, executable components, routines, application programming interfaces (APIs), functions, methods, objects, properties, data structures, data types, and/or the like. Computer-executable instructions may be stored on one or more computer-readable storage media and can be executed by one or more processors, computing devices, and/or computer systems to perform particular tasks or implement particular data types in accordance with aspects of the present invention.

The mass spectrometry system may implement and utilize one or more program modules. Generally, program modules include routines, programs, objects, components, data structures, etc., that perform particular tasks or implement particular abstract data types.

The mass spectrometry system may be implemented as a distributed computing system or environment in which components are located on different computing devices that are connected to each other through network (e.g., wired and/or wireless) and/or other forms of direct and/or indirect connections. In such distributed computing systems or environments, tasks can be performed by one or more remote processing devices, or within a cloud of one or more devices, that are linked through one or more communications networks. In a distributed computing environment, program modules can be located in both local and remote computer storage media including media storage devices. Still further, the aforementioned instructions can be implemented, in part or in whole, as hardware logic circuits, which can include a processor.

The mass spectrometry system may be implemented by one or more computing devices such as computers, PCs, server computers configured to provide various types of services and/or data stores in accordance with aspects of the present invention. Exemplary sever computers can include, without limitation: web servers, front end servers, application servers, database servers, domain controllers, domain name servers, directory servers, and/or other suitable computers.

Components of the mass spectrometry system may be implemented by software, hardware, firmware or a combination thereof. For example, the mass spectrometry system may include components implemented by computer-executable instructions that are stored on one or more computer-readable storage media and that are executed to perform various steps, methods, and/or functionality in accordance with aspects of the present invention.

The mass spectrometry system may include a controller, memory, additional hardware storage, input devices, and output devices. Input devices may include one or more of the exemplary input devices described above and/or other type of input mechanism and/or device. Output devices may include one or more of the exemplary output devices described above and/or other type of output mechanism and/or device, such as a display.

The mass spectrometry system may contain one or more communication interfaces that allow the mass spectrometry system to communicate with other computing devices and/or other computer systems. The mass spectrometry system may include and/or run one or more computer programs implemented, for example, by software, firmware, hardware, logic, and/or circuitry of the mass spectrometry system. Computer programs can include an operating system implemented, for example, by one or more exemplary operating systems described above and/or other type of operating system suitable for running on computing device. Computer programs can include one or more applications.

The terms “circuits” and “circuitry” refer to physical electronic components (e.g., hardware) and any software and/or firmware (“code”) which may configure the hardware, be executed by the hardware, and or otherwise be associated with the hardware. As used herein, for example, a particular processor and memory may comprise a first “circuit” when executing a first set of one or more lines of code and may comprise a second “circuit” when executing a second set of one or more lines of code. As utilized herein, circuitry is “operable” to perform a function whenever the circuitry comprises the necessary hardware and code (if any is necessary) to perform the function, regardless of whether performance of the function is disabled, or not enabled (e.g., by a user-configurable setting, factory trim, etc.).

The terms “communicate” and “communicating” include both conveying data from a source to a destination and delivering data to a communications medium, system, channel, network, device, wire, cable, fiber, circuit, and/or link to be conveyed to a destination. The term “communication” as used herein means data so conveyed or delivered. The term “communications” as used herein includes one or more of a communications medium, system, channel, network, device, wire, cable, fiber, circuit, and/or link.

The terms “connect,” “connected,” and “connection” each mean a relationship between or among two or more devices, apparatuses, files, circuits, elements, functions, operations, processes, programs, media, components, networks, systems, subsystems, and/or means, constituting any one or more of: (i) a connection, whether direct or through one or more other devices, apparatuses, files, circuits, elements, functions, operations, processes, programs, media, components, networks, systems, subsystems, or means; (ii) a communications relationship, whether direct or through one or more other devices, apparatuses, files, circuits, elements, functions, operations, processes, programs, media, components, networks, systems, subsystems, or means; and/or (iii) a functional relationship in which the operation of any one or more devices, apparatuses, files, circuits, elements, functions, operations, processes, programs, media, components, networks, systems, subsystems, or means depends, in whole or in part, on the operation of any one or more others thereof.

The term “data” means any indicia, signals, marks, symbols, domains, symbol sets, representations, and any other physical form or forms representing information, whether permanent or temporary, whether visible, audible, acoustic, electric, magnetic, electromagnetic, or otherwise manifested. The term “data” is used to represent predetermined information in one physical form, encompassing any and all representations of corresponding information in a different physical form or forms.

All documents cited herein are incorporated herein by reference, but only to the extent that the incorporated material does not conflict with existing definitions, statements, or other documents set forth herein. To the extent that any meaning or definition of a term in this document conflicts with any meaning or definition of the same term in a document incorporated by reference, the meaning or definition assigned to that term in this document shall govern. The citation of any document is not to be construed as an admission that it is prior art with respect to this application.

While particular embodiments have been illustrated and described, it would be obvious to those skilled in the art that various other changes and modifications may be made without departing from the spirit and scope of the invention. Those skilled in the art will recognize or be able to ascertain using no more than routine experimentation, numerous equivalents to the specific apparatuses and methods described herein, including alternatives, variants, additions, deletions, modifications and substitutions. This application including the appended claims is therefore intended to cover all such changes and modifications that are within the scope of this application.

What is claimed is:

1. A method of analyzing a feline tissue sample comprising:
(a) generating sample ions directly from the feline tissue sample using a MALDI ionization source;
(b) receiving the ions into a mass spectrometer;
(c) identifying at least one of a feline inflammatory bowel disease related compound and a feline small cell lymphoma related compound in the feline tissue sample from results from the mass spectrometer;
(d) comparing the related compound in the feline tissue sample to one or more known feline inflammatory bowel disease profiles and feline small cell lymphoma profiles; and
(e) identifying at least one condition related to the feline tissue sample from the comparison of the at least one identified related compound to the one or more known profiles.

2. The method of claim 1, wherein the one or more known profiles comprise at least one of m/z 610.3883±0.1; m/z 627.4335±0.1; m/z 659.4198±0.1; m/z 678.3355±0.1; m/z 733.4526±0.1; m/z 802.448±0.1; m/z 943.6057±0.1; m/z 971.5833±0.1; m/z 1068.6376±0.1; m/z 1082.629±0.1; m/z 1093.5984±0.1; m/z 1096.6205±0.1; m/z 1105.553±0.1; m/z 1243.6573±0.1; m/z 1270.76225±0.1; m/z 1402.73355±0.1; m/z 1469.73305±0.1; and m/z 1953.9388±0.1.

3. The method of claim 1, wherein the at least one condition comprises at least one of feline inflammatory bowel disease and feline small cell lymphoma.

4. The method of claim 1 comprising determining a distribution of the one or more analytes within the feline tissue sample.

5. The method of claim 1 comprising performing a pathological comparison of the feline tissue sample with one of a plurality of reference standards for feline inflammatory bowel disease and a plurality of reference standards for feline small cell lymphoma.

6. The method of claim 5, wherein the reference standards differ from each other with respect to at least one of time in cell development, stage of disease progression, culturing or growth conditions, cell type, tissues type, and disease type.

7. The method of claim 5, wherein the pathological comparison includes at least one of immunohistochemistry (IHC), immunofluorescence (IF), hematoxylin and eosin (H&E) staining, clonality testing, and imaging using one of a light microscope, a fluorescence microscope, a confocal microscope, and an electron microscope.

8. The method of claim 1 comprising:
producing a mass spectral image of the feline tissue sample based on results from the mass spectral analysis;
performing a histochemistry analysis technique on the feline tissue sample to thereby stain the sample;
producing an optical image of the stained sample;
overlaying the mass spectral image with the optical image of the stained sample to produce an overlaid image; and
determining a distribution of the one or more analytes in the tissue sample based on an analysis of the overlaid image.

9. The method of claim 8 comprising detecting cancerous tissue within the tissue sample based on an analysis of the overlaid image.

10. The method of claim 1 comprising experimentally measuring a mass to charge (m/z) ratio of the sample ions using the mass spectrometer.

11. The method of claim 4, wherein the mass spectrometer comprises one of a quadrupole mass spectrometer, a time of flight mass spectrometer, an Orbitrap mass spectrometer, and an ion trap mass spectrometer.

12. The method of claim 1 comprising ionizing the feline tissue sample using laser energy to generate the sample ions directly from the feline tissue sample.

13. The method of claim 1, wherein identifying at least one related compound in the feline tissue sample from results from the mass spectrometer comprises generating a mass spectrometry profile of the feline tissue sample from results from the mass spectrometer.

14. The method of claim 1 characterized by a sensitivity and a specificity of at least 85% in correctly classifying the feline tissue sample as one of feline inflammatory bowel disease and feline small cell lymphoma.

15. The method of claim 1 comprising staining the feline tissue sample prior to generating sample ions directly from the feline tissue sample using a MALDI ionization source.

16. The method of claim 1 comprising staining the feline tissue sample after generating sample ions directly from the feline tissue sample using a MALDI ionization source.

17. The method of claim 1 comprising staining the feline tissue sample with hematoxylin and eosin.

18. The method of claim 1 comprising:
treating the feline tissue sample to deparaffinization and antigen retrieval prior to on-tissue tryptic digestion and matrix application using a robotic sprayer;
treating the feline tissue sample to on-tissue tryptic digestion; and
applying a MALDI compatible matrix to the treated feline tissue sample using a robotic sprayer.

19. The method of claim 1, wherein the feline tissue sample comprises a first section and a second section, and wherein the method comprising:
annotating an image of the first section to identify areas of interest on the feline tissue sample having a diameter from 10-400 micrometers; and
imposing the annotated image of the first section upon the second section prior to generating sample ions directly from the areas of interest identified on the second section of the feline tissue sample using the MALDI ionization source.

20. The method of claim 19, wherein the areas of interest are identified as one of feline inflammatory bowel disease and feline small cell lymphoma using histologic analysis and/or immunohistochemical analysis.
